(19) United States
(12) Patent Application Publication
(10) Pub. No.: US 2022/0164816 A1
(43) Pub. Date: May 26, 2022

(54) SYSTEM AND METHOD OF PROVIDING PATIENT DISCOUNT PROGRAMS

(71) Applicant: First Access, Inc., Irvine, CA (US)

(72) Inventor: Kimberly A. Darling, Trabuco Canyon, CA (US)

(21) Appl. No.: 17/496,047

(22) Filed: Oct. 7, 2021

Related U.S. Application Data
(63) Continuation of application No. 17/358,451, filed on Jun. 25, 2021, now abandoned.
Application No. 17/358,451 is a continuation of application No. 17/211,571, filed on Mar. 24, 2021, now abandoned.
Application No. 17/211,571 is a continuation of application No. 16/530,062, filed on Aug. 2, 2019, now abandoned.
Application No. 16/530,062 is a continuation of application No. 15/380,879, filed on Dec. 15, 2016, now abandoned.
(60) Provisional application No. 62/387,566, filed on Dec. 23, 2015.

(51) Int. Cl. G06Q 30/02; G16H 40/20; G06Q 10/10

(52) U.S. Cl. CPC G06Q 30/0207; G06Q 10/10; G16H 40/20

(57) ABSTRACT

A medical system is described that provides discounted prices for medical services. A discount network includes discount programs for medical providers, wherein each of the medical providers is identified by a unique identifier of a card reader. A transaction processing system provides transaction information relating to a first card transaction, wherein the transaction information includes the unique ID of the card reader, a card number, and an amount charged in the transaction. The medical system accesses one or more databases to determine whether a discount program exists for the unique ID of the card reader. In response to determining that a discount program exists for the unique ID of the card reader, the medical system automatically performs a second card transaction to subtract a second amount that reflects the discount for the medical procedure.

### INCORPORATION BY REFERENCE TO ANY PRIORITY APPLICATIONS

Any and all applications, if any, for which a foreign or domestic priority claim is identified in the Application Data Sheet of the present application are hereby incorporated by reference under 37 CFR 1.57.

### BACKGROUND

Medical service providers, such as a hospital or a doctor's office, may offer discounts for services or procedures they provide. Discount providers may also administer one or more discount networks through which medical service providers offer discounts.

### SUMMARY

In some cases, medical service providers may want to provide discounts for services they provide if patients are eligible. For example, a company can create discount programs under agreements with medical providers for some or all services provided by respective medical providers under agreements. Patients can choose to enroll in these discount programs. However, application of the relevant discounts and processing of the discounts during a payment transaction can become cumbersome. For example, an administrator of the medical service provider may need to call the company to find out how much discount applies or whether a patient is eligible to receive a discount.

In order to address these and other challenges, a system according to certain aspects provides discounts for medical services that are integrated into payment transactions for the medical services. The integrated discounts can eliminate or reduce human involvement in applying and/or processing the discounts, thereby streamlining the process.

For purposes of summarizing the disclosure, certain aspects, advantages and novel features of the inventions have been described herein. It is to be understood that not necessarily all such advantages may be achieved in accordance with any particular embodiment of the invention. Thus, the invention may be embodied or carried out in a manner that achieves or optimizes one advantage or group of advantages as taught herein without necessarily achieving other advantages as may be taught or suggested herein.

### BRIEF DESCRIPTION OF THE DRAWINGS

FIG. 1 is a block diagram illustrating an exemplary system for providing patient discounts for medical services.

FIG. 2 is a data flow diagram illustrative of the interaction between the various components of an exemplary system configured to provide discounts for medical services transactions, according to certain embodiments.

FIG. 3 is a data flow diagram illustrative of the interaction between the various components of an exemplary system configured to provide discount programs for medical services, according to certain embodiments.

FIG. 4 is a flow diagram illustrative of a routine for providing discounts for medical service transactions, according to certain embodiments.

FIG. 5 is a flow diagram illustrative of a routine for providing discount programs for medical services, according to certain embodiments.

### DETAILED DESCRIPTION

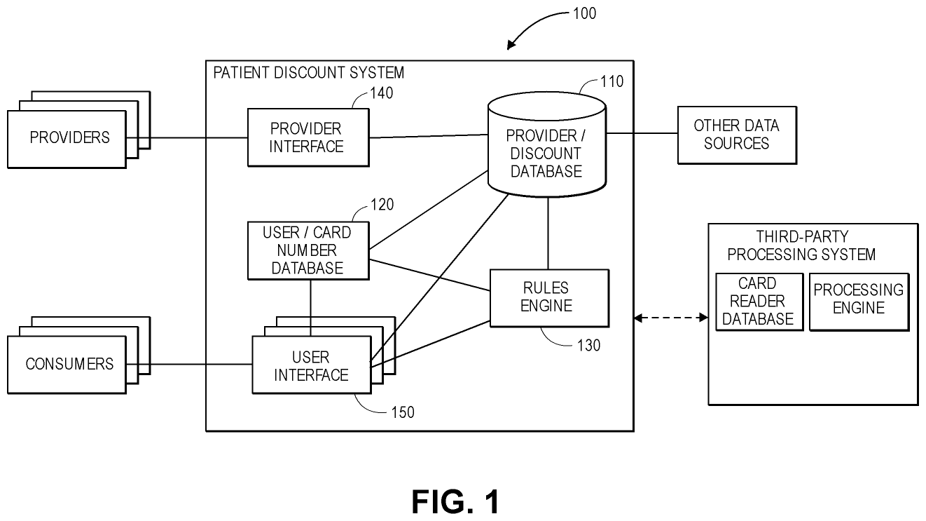

Systems and methods are disclosed for implementing discounts for medical services. Examples of such systems and methods are described in further detail herein, in reference to FIGS. 1-5.

FIG. 1 is a block diagram illustrating an exemplary system 100 for providing patient discounts for medical services. A discount provider can implement the system 100 in order to provide and process discounts for medical services performed by various providers. For example, the discount provider creates and manages one or more discount networks or programs in which users can enroll in order to receive discounts for medical services from specific providers. The discount provider also processes discounts applied to services performed by the specific providers. A provider may refer to an entity that provides medical or related services, such as a hospital, a doctor's office, a physical therapist's office, etc. The techniques of the disclosure are explained in context of medical services and medical service providers for illustrative purposes. These techniques can also be applicable to health services and health service providers as well as other types of services and other types of providers.

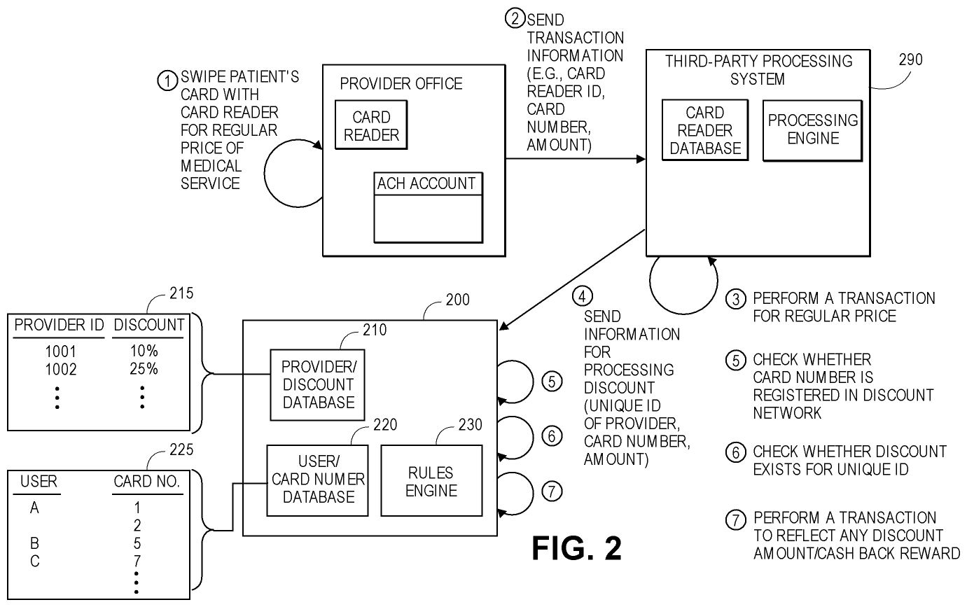

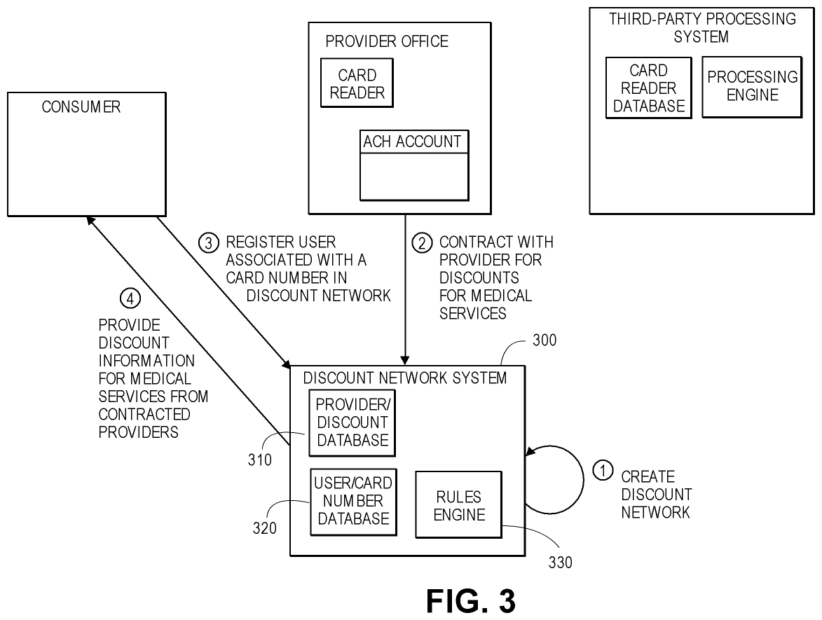

In the example of FIG. 1, the system 100 includes a provider/discount database 110 that contains discount information for various providers. For example, the provider/discount database indicates a unique identifier (ID) associated with a provider (e.g., a merchant ID or terminal ID) and one or more discounts offered by the provider. The unique ID associated with the provider may also be referred to as the provider unique ID. The unique ID will be explained further below, e.g., in connection with FIGS. 2 and 3. The provider/discount database 110 can also include information about the discount networks or programs offered by the discount provider. In one example, the discount provider may offer two different discount networks, one for dental services and one for dermatology services. The provider/discount database 110 can maintain information regarding which unique IDs belong to which network or program. The system 100 also includes a user/card number database 120 that contains information about users and/or card numbers enrolled in a discount network program. For instance, the user/card number database 120 indicates the user and one or more card numbers associated with the user. The card number can be a number associated with a credit card, a debit card, a reloadable card, a single use card, a virtual card, a gift card, etc. The provider/discount database 110 and/or the user/card number database 120 may include one or more data structures for storing information, such as tables, files, data objects, etc.

The system 100 can also include a rules engine 130 that processes the discounts. When a user makes a payment using a registered card number for a service by a provider, a third-party processing system processes the payment. When processing the payment, the third-party processing system passes to the system 100 information used for processing discounts, such as the unique ID of the provider and the card number. The rules engine 130 may then access the provider/discount database 110 to check whether the unique ID has any discounts associated with it. If at least one discount exists, the rules engine 130 applies the discount appropriately for the card number.

In the example of FIG. 1, the system 100 also includes a provider interface 140 and a user interface 150. Providers can interact with the system 100 through the provider interface 140. For instance, providers can register in one or more discount networks and specify the discounts they wish to offer. Providers can also view information about the discount networks they are a part of and the discounts they offer through the discount networks. Users may interact with the system 100 through the user interface 150. Users can register in one or more discount networks and enter one or more card numbers in their profiles. Users may also view information relating to the discount networks they are registered in, the discounts available, the discounts applied, etc. The provider interface 140 and/or the user interface 150 may be provided or implemented in various forms depending on the embodiment. For example, the provider interface 140 and/or the user interface 150 can be a web page, an application, a mobile app, by phone, by fax, by email, etc. The system 100 is provided for illustrative purposes and can include additional and/or different components as appropriate, depending on the embodiment.

FIG. 2 is a data flow diagram illustrative of the interaction between the various components of an exemplary system 200 configured to provide discounts for medical services transactions, according to certain embodiments. The system 200 and corresponding components of FIG. 2 may be similar to or the same as the system 100 and similarly named components of FIG. 1. Moreover, depending on the embodiment, the system 200 of FIG. 2 may additionally include any of the other components shown in FIG. 1 that are not specifically shown in FIG. 2. The system 200 may include one or more of each component. All components of the system 200 can be in direct communication with each other or communicate indirectly via other components. In certain embodiments, some of the components in FIG. 2 shown as separate components can reside on a single computing device, or vice versa.

In the example of FIG. 2, a patient visits a provider's office to receive a medical service. The patient is enrolled in one or more discount networks or programs offered by the discount provider. The provider may also be enrolled in one or more discount networks or programs offered by the discount provider.

At data flow step 1, the provider swipes a patient's card with the card reader for the regular price of a medical service. The patient pays with a card that is enrolled in a discount network. The card number can function as a unique ID for the patient for purposes of receiving the discount. Since the patient is also a user of the system 200 and discount networks, the patient may also be referred to as the user in order to facilitate discussion. The user may register multiple cards in the system 200. For example, the user has a user account in the system 200 and enters the numbers of cards for which the user wants to receive discounts. In one embodiment, the system 200 associates the user with specific discount networks. In another embodiment, the system 200 associates each card of the user with specific discount networks. If the user registers multiple cards, the card number of each card can serve as a unique ID for the user.

A card can be issued by a bank, a credit union, or any other appropriate entity. The bank or the entity may be referred to as the issuer, or in the case of the bank, also as the issuing bank. As explained above, different types of cards can be used. Some examples of such cards include: a credit card (e.g., Visa, MasterCard, American Express, Discover, etc.), a debit card, a reloadable card (e.g., a general purpose reloadable (GPR) card), a single use card, a virtual card, a gift card, a close network or house card (e.g., department store specific or store specific card, etc.), etc. A reloadable card can refer to a card that can be reloaded with additional balance. The card number of a reloadable card can be used like a credit card and be used in various transactions, such as auto payment. A GPR card can be used for various purposes like a credit card. A single use card may refer to a card that can only be used for the stored value of the card (e.g., $100 Visa prepaid card). The user may not add additional value to the card. A virtual card can refer to a card that does not physically exist but has a card number that can be used in transactions. A gift card may refer to a card that has a stored value and may be used at a particular store or chain (e.g., a department store). A close network or house card may refer to a card that functions like a credit card but is only accepted at a particular store or chain. Although these techniques are described in the context of using cards, any other technology that functions in the same or a similar manner as cards can be utilized.

Various types of technology can be used to execute payment transactions. Some examples of such technology include magnetic stripes, integrated circuits (ICs) or chips, radio frequency identification (RFID), near field communication (NFC), beacon, etc. A beacon may refer to hardware that uses low energy Bluetooth connections to transmit messages. Beacon technology can enable a smart phone or other devices to perform actions when the devices are in close proximity to a beacon (e.g., make payments, etc.). A card reader may refer to any equipment or machine that is used to accept a payment using a card. For example, a card reader can be a credit card terminal (e.g., for reading magnetic stripe cards), an embedded chip terminal, an RFID reader, an NFC reader, etc. A particular card reader may be compatible with one or more types of technologies. For instance, a card reader can read both magnetic stripe cards and embedded chip cards. Each card reader can have a unique ID associated with it. For instance, a credit card terminal has a terminal ID that uniquely identifies that particular terminal. The provider can obtain a card reader from a bank or another entity under an agreement for accepting payments using cards. The provider may set up an account with the bank or the entity (or a third party designated by the bank or the entity), which may be referred to as the merchant account. Under the agreement, the provider can also receive a merchant ID that uniquely identifies the provider. In order to facilitate discussion, the provider may also be referred to as a merchant. The bank or the entity with which the merchant has the agreement may be referred to as the acquirer, or in the case of the bank, also as the acquiring bank.

In the example of FIG. 2, the provider can swipe the patient's card or enter the card number manually at the card reader in order to initiate a payment transaction. The provider can enter the amount to be charged to the card, then swipe the card or enter the card number to start a transaction for the entered amount using the card. Swiping the card is described for illustrative purposes, and any other action that allows the provider to use the card or recognizes the card to initiate a payment transaction can be used. For example, the provider can hold a card near a terminal. In certain embodiments, a token that is encrypted can be used. Any type of action that can be taken with respect to a card in order to obtain a payment identifier can be used.

At data flow step 2, the card reader sends transaction information to a third-party processing system 290. In one embodiment, transaction information includes the card reader ID, the card number, and the amount to be charged. A third-party processing system 290 may be associated with an entity that processes payment transactions using various types of cards. In some embodiments, the entity may be referred to as a processor. The merchant's agreement with the acquiring bank or another entity may designate a processor that handles the processing of payment transactions performed using the card reader. For example, if a card is swiped at the card reader, the card reader contacts the third-party processing system 290 and sends the transaction information to the third-party processing system 290, and the third-party processing system 290 communicates with the issuer to obtain authorization for the transaction. If the issuer authorizes the transaction, the provider can proceed with the payment transaction.

In one embodiment, the third-party processing system 290 includes a card reader database 291 and a processing engine 292. The card reader database 291 may include information about card readers, such as the unique ID, and the processing engine 292 can process transactions from merchants.

At data flow step 3, the third-party processing system 290 performs a transaction for the regular price of the medical service. As mentioned above, when the third-party processing system 290 receives the transaction information from the card reader, the third-party processing system 290 contacts the issuer to obtain authorization for the transaction, and if the transaction is authorized, performs the transaction. This process may be referred to as authorization.

The transaction information can be stored by the merchant for a period of time (e.g., a day) and sent to the acquirer. For example, the merchant stores transaction information of all transactions for a particular day and submits the transaction information to the acquirer at the end of the day, e.g., as a batch. This process may be referred to as batching. Or the merchant can submit one or more transactions to the acquirer in real-time or close to real-time. For instance, the merchant submits the transaction information for each transaction after it is completed at the card reader. Or if a few transactions occur within a short period of time (e.g., within minutes of each other), the merchant can submit these transactions together to the acquirer. Some or all functions performed by the acquirer may be performed by the processor on behalf of the acquirer. For example, the merchant may submit the transactions to the third-party processing system 290, which processes the transactions on behalf of the acquirer.

After receiving the transactions from the merchant, the acquirer or the third-party processing system 290 can send the transactions to a card network. A card network, such as Visa and MasterCard, acts as an intermediary between issuers and acquirers. Then, the card network sends the transactions to the corresponding issuers. The issuer subtracts interchange fees and transfers the remaining amount to the acquirer. Interchange fees may be set by the card network and shared between the card network and the issuer. This process may be referred to as clearing and settlement. In some embodiments, the discount network or program is not associated with a card network; in such case, the transactions are not sent through a card network.

After clearing and settlement, the acquirer subtracts the discount rate from the amount received from the issuer and pays the merchant the remaining amount. Discount rate or fee may refer to a processing fee paid by the merchant to the acquirer. The issuer bills the cardholder for the amount of the payment transaction. This process may be referred to as funding.

At data flow step 4, the third-party processing system 290 sends information used for processing discounts to the system 200. The information for processing discounts may be referred to as discount processing information. The third-party processing system 290 may send the discount processing information as a part of processing the payment transaction as explained above. The discount provider can set up a discount network (or program) and create rules for the discount network (or program). The discount provider may obtain approval from an issuer for the network. If the network uses a card network, such as Visa or MasterCard, the discount provider may also obtain approval from the card network. An entity that creates and manages the discount network may be referred to as a program manager. For instance, a program manager implements and administers the discount network. When the discount provider creates the discount network, the discount provider acts as the program manager. In other embodiments, another entity can create the discount network on behalf of the discount provider and act as the program manager. For example, the processor can be the program manager, in addition to being the processor. In some cases, the discount provider creates and manages a discount network for a specific client or entity, such as a company or a store. In other cases, the discount provider creates and manages a general discount network in which any user can enroll.

A program manager can obtain or receive transaction information associated with the discount network or program from the third-party processing system 290, e.g., through application programming interfaces (APIs). The transaction information can include discount processing information. Or the third-party processing system 290 can extract discount processing information from the transaction information and make it available to the program manager. In some embodiments, the third-party processing system 290 may also send the transaction information to other entities related to transaction(s), such as the issuer, the merchant, etc. When a card is used to make a payment (e.g., by a swipe, entering the card number, tapping the card, etc.), one or more entities related to the payment transaction can be informed about the payment transaction, such as the issuer, card network, processor, program manager, client, etc.

The system 200 may obtain or receive the transaction information or the discount processing information from the third-party processing system 290. For example, the rules engine 230, or another component of the system 200, receives the discount processing information and accesses relevant information in the discount processing information in order to apply any relevant discounts. The discount processing information may include the unique ID of the provider and the card number of the patient. In one embodiment, the unique ID of the provider is the card reader ID used for the payment transaction. For example, the provider may have one card reader, so the card reader ID can unique identify the provider. In another embodiment, the unique ID of the provider is the merchant ID of the provider. The provider may have multiple card readers, so the system 200 can associate a discount with the merchant ID, instead of associating it with each card reader ID.

At data flow step 5, the system 200 checks whether the card number is registered in a discount network. The rules engine 230 may first check whether the card number in the discount processing information is registered in a particular discount network. The rules engine 230 can refer to the user/card number database 220. The user/card number database 220 may include or may be organized using one or more data structures 225, such as one or more tables, files, data objects, etc. For example, the user/card number database 220 includes a table that lists users and card numbers associated with the users. A user may have multiple card numbers associated with the user. In the example of FIG. 2, the data structure(s) 225 shows that User A has card numbers 1 and 2, User B has card number 5, User C has card number 7, etc. The card numbers in the example of FIG. 2 are simplified for illustrative purposes, and any type or format can be used for the card numbers.

In some embodiments, the system 200 does not check whether the card number is registered in a discount network; the system 200 can know from receiving the discount processing information for a card that the card is a registered card. For instance, the third-party processing system 290 would not send the discount processing information for a card unless the card is registered in at least one discount network provided by the system 200.

At data flow step 6, the system 200 checks whether a discount exists for the unique ID. The rules engine 230 or another component can access the provider/discount database 210 to determine if any discount exists for the unique ID. The provider/discount database 210 may include or may be organized using one or more data structures 215, such as one or more tables, files, data objects, etc. For example, the provider/discount database 210 can include one or more tables that specify discounts offered by providers registered in a discount network or program. In some embodiments, information about discounts offered by providers is maintained as one or more files, in addition to or instead of tables. In the example of FIG. 2, the data structure(s) 215 shows that Provider ID 1001 has a 10% discount, Provider ID 1002 has a 25% discount, and so forth.

The system 200 can provide different types of discounts or specify discounts in a number of different ways. In one embodiment, the unique ID of the provider is associated with a discount percentage to be applied to the amount charged in a payment transaction. For example, the card reader ID is used as the unique ID of the provider, and the provider/discount database 210 includes a table that lists the card reader ID of the provider and a corresponding discount percentage offered by the provider. The third-party processing system 290 provides the card reader ID of the provider in the discount processing information, and the rules engine 230 accesses the provider/discount database 210 using the card reader ID to look up the discount percentage associated with the particular card reader ID. For instance, Doctor A's card reader ID is 1001, and the rules engine 230 checks the provider/discount database 210 and determines that the discount percentage for card reader ID 1001 is 10%.

In other embodiments, a combination of the provider unique ID and the card number can be used to determine the discount amount. The digits in the card number convey information about the issuer and the card network associated with the card. The first digit in a credit card number indicates the card network, for example, whether the credit card is a Visa card, Master card, etc. The next few digits indicate the issuer, such as a bank, a credit union, etc. A provider may use the information about the card network, the issuer, and/or other information in defining discounts. For instance, a provider may distinguish between the card network and/or the issuer. In one example, a user who has a Visa card from Bank A receives a 30% discount, and a user who has MasterCard card from Bank A receives a 15% discount. Or a user who has a Visa card from Bank B receives a 25% discount. The provider could have an agreement with a particular card network and/or a particular issuer that allows the provider to offer higher discounts to users that have a card associated with the particular card network and/or the particular issuer. In such cases, a table in the provider/discount database 210 can list the provider unique ID, the card network number of ID and/or the issuer number or ID, and the discount percentage. There can be multiple entries for the same provider unique ID. In this way, the system 200 can provide multi-tier discounts, for example, different levels of discounts for services provided by the same provider. In some embodiments, the provider offers only one discount based on the card number; for example, the provider offers one discount only for a particular card network and/or issuer, instead of offering different discounts for different card networks and/or issuers.

In certain embodiments, the type of card is associated with a discount percentage. The provider/discount database 210 can specify the discount percentage for a particular type of card that is offered by a provider. The type of card can be indicated by the card number, type of technology used for the card (e.g., RFID, NFC, etc.), etc. The discount processing information may include information about the type of the card used in the transaction, so the rules engine 230 can identify the discount for the type of card used. In one example, a provider decides to offer a 30% discount for an American Express Platinum Card, and the rules engine 230 checks whether a card is an American Express Platinum Card by referring to card chip information included in the discount processing information, if any. A table in the provider/discount database 210 can indicate the provider unique ID, the type of card, and the discount percentage. In this way, the provider can vary the discount between different types of cards.

A provider unique ID may have one discount associated with it or multiple discounts associated with it, depending on the embodiment. The type of discount can be any of the different types of discounts discussed above. If more than one discount is associated with a provider unique ID, the different types of discounts discussed above may be used in combination. For instance, a provider offers one discount that specifies one discount percentage that applies to all cards. Or a provider offers two discounts that each specify a different discount percentage based on a combination of the provider unique ID and the card type. In another example, a provider offers two discounts: one discount that specifies a discount percentage that applies to all cards as the default, and another discount that is based on a combination of the provider unique ID and the card type, which can supersede the default discount.

If a provider unique ID is associated with more than one discount, the system 200 can determine which discount to apply to the transaction. In some embodiments, the system may determine that none of the discounts applies to the transaction, e.g., because the type of card does not qualify for a discount.

At data flow step 7, the system 200 performs a transaction to reflect any discount amount/cash back reward. If the card reader is registered in a discount network or program, and a discount associated with the provider unique ID exists in the same discount network or program, the system 200 can reflect the discount amount. Under an agreement with the provider, the discount provider may subtract the discount amount from the charged amount for the initial transaction. In one embodiment, the system 200 subtracts the discount amount from the amount charged in the initial transaction by performing a second card transaction. For example, the amount charged for the service by the provider in the initial transaction is credited to an account of the provider (e.g., an automated clearing house (ACH) account), and under authorization of the provider, the system 200 subtracts the discount amount from the ACH account of the provider and adds the discount amount to an account of the user. The system 200 may have account information for users (e.g., in the user profile) or may receive the account information from the issuer, depending on the embodiment.

The system 200 may provide a refund in any appropriate form or manner, other than applying the discount amount in a second card transaction. The user may select the form of the refund or indicate preference using the system 200. In one embodiment, the system 200 issues a gift card to the user in the discount amount. The gift card can be associated with a particular store or a particular card network (e.g., Visa debit card). In other embodiments, the system 200 can provide miles, points, cash back, checks, etc. for the refund amount. Or the system 200 may sell gift cards at a discounted rate reflecting the refund amount. If the refund due to the user is $20, the user can purchase a $100 gift card from the system 200 for $80. In some embodiments, the provider sells the gift cards at a discounted rate, and the user can purchase the gift card from the provider; in the previous example, the user can purchase the $100 gift card for $80 from the provider. Or the user may purchase a service by the provider, such as an office visit, at a reduced rate (e.g., online, on a mobile device, etc.). The system 200 may subtract the amount to be refunded from the account of the provider or receive a payment from the provider for the amount of the refund, depending on the embodiment. For example, if the discount provider issues a gift card to the user as a refund, the provider authorizes the discount provider to subtract the refund amount from the provider's account or pays the discount provider for the gift card at a later time. In some embodiments, the system 200 can use various types of payment channels or systems, such as Apple Pay, PayPal, iPad kiosk, gift card malls, loyalty programs, insurance carrier portals and applications, etc., for example, in order to provide discounts to the user or to receive any payments from the provider.

The discounts can be applied in batch. For example, after the provider submits all the transactions for a particular day in a batch, the system 200 can apply the discounts to any relevant transactions from the batch for which the third-party processing system 290 sends the discount processing information. Or the discounts may be applied as the provider submits each transaction or a group of transactions in real-time or close to real-time. The third-party processing system 290 can send the discount processing information for each transaction or group of transactions as the provider submits them.

In this manner, the system 200 can automatically identify and apply any relevant discounts for a medical service provided by the provider when the user makes a payment using a registered card. Once a payment transaction is initiated for a particular amount, it may be difficult to adjust the amount to be charged within the same transaction to reflect the discount amount, for example, without changing the credit card workflow and the third-party processing system 290. For instance, the third-party processing system 290 would implement a mechanism to obtain discount information from the system 200 during the authorization process and to change the amount to be charged from the amount that was entered to initiate the payment transaction. In addition, the card networks may need to approve such implementation and changes to the workflow. Using the system 200, any discounts can be integrated into the payment in the sense that the provider and/or the user does not take any additional steps other than the act of making a payment in order to receive the discount. Employees of the provider do not have to find out whether a card qualifies for a discount or be aware of any discounts when swiping a card. The user does not have to keep track of whether a particular provider offers discounts and simply use a registered card in order to receive relevant discounts. Also, the user does not have to present any additional cards other than the card with which the user is making a payment.

Now an illustrative example will be described with reference to FIG. 2. User A visits Dr. Smith's office and receives a service. The regular price of the service User A receives is $50. User A is a registered member of Discount Network 1 provided by the discount provider and has two cards listed in the user profile, Card 1 and Card 2. Dr. Smith offers a 10% discount to all cards registered in Discount Network 1. User A decides to use Card 1 to make a payment for the service at Dr. Smith's office. Dr. Smith's office has one card reader, and the card reader has a card reader ID, which is a unique ID that identifies the card reader. The employee at Dr. Smith's office enters the amount to be charged, $50, at the card reader and swipes Card 1. When Card 1 is swiped at the card reader, the card reader contacts the third-party processing system 290, which then contacts the issuer of Card 1 to obtain authorization for the transaction. The issuer of Card 1 authorizes the transaction, and the third-party processing system 290 forwards the authorization information to the card reader, and the transaction is successful.

Dr. Smith's office stores all transactions performed using the card reader until the end of the business day and submits the stored transactions to the third-party processing system 290 in a batch at the end of the business day. The third-party processing system 290 receives the batch and forwards the transactions to one or more appropriate card networks. The card networks forward the transactions from the third-party processing system 290 to the appropriate issuers for clearing and settlement. The third-party processing system 290 sends the transaction information or discount processing information for various transactions to the system 200 (e.g., through an API). The system 200 receives discount processing information for User A's transaction from the third-party processing system 290 as a part of this process. The discount processing information for User A's transaction can include the unique ID of Dr. Smith's card reader, the card number of Card 1, and the amount charged in the transaction, $50.

When the system 200 receives the discount processing information for User A's transaction, the system 200 checks whether Card 1 is registered in any of the discount networks provided by the discount provider. The system 200 determines from referring to the user/card number database 220 and/or the provider/discount database 210 that Card 1 is registered in Discount Network 1. Then, the system 200 accesses the provider/discount database 210 to check whether any discount associated with the card reader ID exists. The provider/discount database 210 indicates that for the unique ID of Dr. Smith's card reader, the discount percentage is 10%. Since Card 1 is registered in Discount Network 1 and Dr. Smith offers a discount under Discount Network 1, the system 200 applies the discount to User A's transaction and performs a separate transaction to credit the discount amount to User A's account. The system 200 subtracts the discount amount, $5, from Dr. Smith's account and adds the discount amount to User A's account.

If User A uses Card 1 at a provider that is not a part of the discount network (e.g., Discount Network 1), User A is charged the regular price of the medical service, and the system 200 does not perform a subsequent transaction to reflect any discount amount. Similarly, if User A uses Card 1 at a provider that does not belong to the same discount network as User A, User A does not receive a discount. For example, if Dr. Smith provides a discount under Discount Network 2 and not Discount Network 1, User A may not receive any discount from Dr. Smith unless User A also has cards registered in Discount Network 2.

The techniques for providing discounts have been described in the context of medical services for illustrative purposes, and these techniques can be used for any type of services to which a discount can be applied as a part of the payment transaction. Also, the services are not limited to those provided by medical providers. Some examples of other services can include: gas station services, automobile services, dry cleaning services, etc. The techniques may apply to different types of medical or medical-related services, such as dental, vision, behavioral health, imaging, lab, alternative care, etc.

FIG. 3 is a data flow diagram illustrative of the interaction between the various components of an exemplary system 300 configured to provide discount programs for medical services, according to certain embodiments. The system 300 and corresponding components of FIG. 3 may be similar to or the same as the system 100, 200 and similarly named components of FIGS. 1 and 2. Moreover, depending on the embodiment, the system 300 of FIG. 3 may additionally include any of the other components shown in FIGS. 1 and 2 that are not specifically shown in FIG. 3. The system 300 may include one or more of each component. All components of the system 300 can be in direct communication with each other or communicate indirectly via other components. In certain embodiments, some of the components in FIG. 3 shown as separate components can reside on a single computing device, or vice versa.

FIG. 2 describes certain details of the process of applying discounts for medical services for users and providers that are registered in a discount network provided by the discount provider. It may also be useful to provide a system that provides discount information to users and allows providers to create discount programs. Accordingly, the system 300 can implement features for creating discount programs and providing discount information. The users and providers can register in one or more discount networks using the system 300.

At data flow step 1, the system 300 creates a discount network. The system 300 can create one or more discount networks in which providers can register to offer discounts for their services. The system 300 can many different types of discount networks, e.g., specified by type of service, type of industry, etc. In one example, the system 300 has a discount network for clothing retailers and another discount network for doctors. In some embodiments, the system 300 has multiple discount networks within a type of service, type of industry, etc. For instance, for medical services, the system 300 may have a discount network for primary physicians, a discount network for dentists, a discount network for dermatologists, etc.

At data flow step 2, the discount provider contracts with a provider for discounts for medical services. For example, providers that want to offer discounts through the discount network sign an agreement with the discount provider in order to authorize transfer of the discount amounts from the providers to the users. The providers can register in one or more discount networks in order to provide discount programs.

The system 300 can provide a provider interface, which can be the same as or similar to the provider interface 140 in FIG. 1. Through the provider interface, the providers may register in a discount network, create a discount program, view transactions associated with discounts, etc. The provider interface can be provided in various forms, e.g., as web pages, as an application, as a mobile app, by email, by phone, by fax, by mail, etc.

A provider can create a discount program in a particular discount network. A discount program may refer to any discounts that a provider offers through a particular discount network. In some embodiments, a discount program may refer discounts that a provider offers through multiple networks; a discount program can span across different networks. A discount program can specify one or more discounts for services performed by the provider. As explained above, the discount can be a blanket discount that applies the same amount to all transactions. Or a discount program can have multiple discounts based on the type of card, the card network, the issuer, any combination thereof, etc. For instance, the provider defines different discount percentages for specific banks or specific card networks. As discussed above, such discount may be identified based on the provider unique ID, and issuer number/ID or card network number/ID. Information relating to providers and networks may be stored in the provider/discount database 310, which can be similar to the provider/discount database 110 and 210 in FIGS. 1 and 2. For example, the provider/discount database 310 may store the information in one or more data structures, such as tables, files, data objects, etc.

A provider may update the discount information as appropriate using the system 300. For example, a provider initially sets the discount percentage to 10%, but later changes it to 15%. In some cases, in order to provide advance notice to users, the system 300 may not allow the providers to update the discount rates too frequently. For instance, if a provider makes a change to the discount rate every other day, the users may not be aware what the current discount rate is. Accordingly, in some embodiments, the system 300 places a limit on the frequency of updates to the discount information (e.g., weekly, bi-weekly, monthly, etc.).

At data flow step 3, the system 300 registers a user associated with a card number in the discount network. Similar to the providers, users can register in one or more discount networks in order to receive discounts from registered providers. The user may create a profile in order to register in the discount networks. The user can enter one or more card numbers the user may use to receive discounts. The card numbers can be associated with the user profile. The user can choose to enroll some or all of the user's cards in a particular discount network the user is registered in, depending on the embodiment. For instance, if the user registers two cards and registers in two different discount networks, the user may enroll both cards in both networks, only enroll one in each network, or enroll both cards in one network but only one card in another network, etc. Information relating to users and cards may be stored in the user/card number database 320, which can be similar to the user/card number database 120 and 220 in FIGS. 1 and 2. For example, the user/card number database 320 may store the information in one or more data structures, such as tables, files, data objects, etc.

The system 300 can provide a user interface, which can be the same as or similar to the user interface 150 in FIG. 1. Through the user interface, the users may register in a discount network, register one or more cards, view transactions associated with discounts, etc. The user interface can be provided in various forms, e.g., as web pages, as an application, as a mobile app, by email, by phone, by fax, by mail, etc. In some embodiments, the user interface can use various types of payment options or systems, such as Apple Pay, PayPal, iPad kiosk, gift card malls, loyalty programs, insurance carrier portals and applications, etc.

At data flow step 4, the system 300 provides the discount information for medical services from contracted providers to registered users. The system 300 may provide the discount information through the user interface.

The system 300 may provide features that help the user select a provider. The system 300 may allow the user to search for providers offering certain types of services, price of services within a certain range (e.g., prior to or after applying relevant discounts), etc. Also, if multiple providers offer services for the same price after applying the discounts, the system 300 may recommend a particular provider based on ratings, reviews, etc. The system 300 can also provide other features such as provider information and directions to a provider's office. In some embodiments, the system 300 provides quality and/or outcome ratings, information relating to hospital affiliations, etc.

In this manner, the system 300 can allow users to select a provider to visit based on the discounts the provider offers for medical services. The system 300 also makes up-to-date discount information available to users. The providers can update the discount information using the system 300, and the updates can be available to users in real-time or close to real-time.

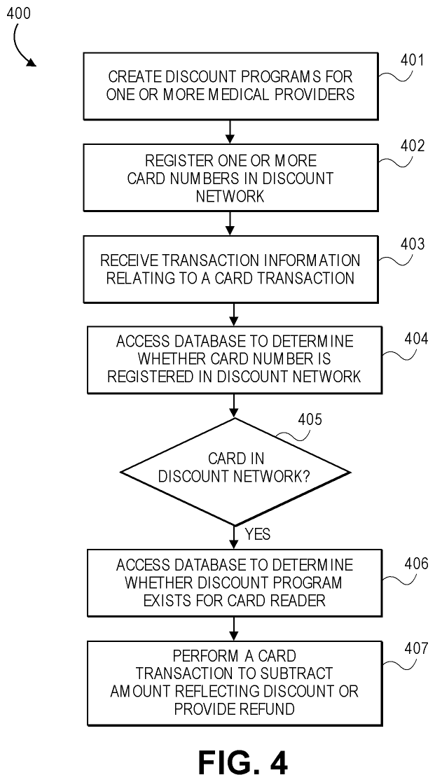

FIG. 4 is a flow diagram illustrative of a routine 400 for providing discounts for medical service transactions, according to certain embodiments. The routine 400 is described with respect to the system 200 of FIG. 2. However, one or more of the steps of routine 400 may be implemented by other systems, such as those described in greater detail above with reference to FIGS. 1 and 3. The routine 400 can be implemented by any one, or a combination of, components in the system 200. Further details regarding certain aspects of at least some of steps of the routine 400 are described in greater detail above with reference to FIG. 2.

At block 401, the system 200 creates discount programs for one or more medical providers. A discount program for a particular medical provider can include one or more discounts that can be applied to services provided by the medical provider. Each of the one or more medical providers can be identified by a unique identifier (ID) of a card reader associated with respective medical provider. A card reader may be configured to perform a card transaction using a card. Each discount program may specify at least one discount for medical procedures provided by respective medical provider. The discount programs may be provided by a discount network. The discount provider creates one or more discount networks, and the discount program(s) of medical providers can be associated with some or all of the discount networks. The card reader associated with the respective medical provider may recognize a card having a magnetic stripe, a RFID chip, a NFC chip, or any combination thereof, etc. Information relating to the discount programs and the discount networks may be stored in one or more data structures in one or more databases (e.g., data structure(s) 215 in the provider/discount database 210).

At block 402, the system 200 registers one or more card numbers in the discount network. Information relating to users and the one or more card numbers may be stored in the one or more data structures in one or more databases (e.g., data structure(s) 225 in the user/card number database 220). At block 403, the system 200 receives transaction information relating to a card transaction. Subsequent to a card transaction using a card reader of a medical provider for a medical procedure provided by the medical provider, the system 200 receives the transaction information relating to the card transaction from a transaction processing system 290. The card transaction may be initiated by a swipe or recognition of a card at the card reader. The card reader may recognize a card in many different ways, for example, by swiping of the card, inserting of the card, tapping the card, placing the card near the card reader, etc. As mentioned above, the card reader can use any technology that can provide a payment identifier for a transaction using the card.

The transaction information may include the unique ID of the card reader, the card number of the card used in the card transaction, and an amount charged in the card transaction. The card may be a credit card, a debit card, a gift card, a general purpose reloadable (GPR) card, a single use card, a virtual card, a closed network card, or any combination thereof, etc. In one embodiment, the system 200 receives the transaction information relating to the card transaction from the transaction processing system 290 in real-time. In another embodiment, the system 200 receives the transaction information relating to the card transaction from the transaction processing system 290 at a time subsequent to the time at which the card transaction is performed. For example, when the transaction processing system 290 submits the batch for a day and the transactions in the batch are forwarded to the appropriate issuer by card networks, the system 200 may receive at least some of the transactions as a group.

At block 404, the system 200 accesses a database to determine whether the card number is registered in the discount network. In one embodiment, the database is the user/card number database 220 or the provider/discount information database 210.

At block 405, if the card number is in the discount network, the system 200 accesses the database to determine whether a discount program exists for the card reader, at block 406. In one embodiment, the database is the provider/discount information database 210.

At block 407, if a discount program exists for the card reader, the system 200 performs a card transaction to subtract an amount reflecting the discount or provides a refund. The card transaction at block 407 may be referred to as a second card transaction in order to distinguish it from the card transaction at block 403. The system 200 can perform the second card transaction automatically. The system 200 subtracts the amount reflecting the discount from an account of the medical provider to an account of the user associated with the card number. Or the system 200 initiates or provides a refund for the amount reflecting the discount in a form indicated in association with the card number. The system 200 can initiate or provide the refund automatically. The amount reflecting the discount may be referred to as a second amount in order to distinguish it from the amount charged in the card transaction at block 403. In certain embodiments, the system 200 provides the refund for the second amount by providing points associated with a reward program, mileage associated with a reward program, a cash back amount, a check, a gift card, or any combination thereof, etc.

In one embodiment, the discount in the discount program for the unique ID of the card reader is a discount percentage associated with the unique ID, where the discount percentage is to be applied to an amount charged in a card transaction. In another embodiment, the discount in the discount program for the unique ID of the card reader is a discount percentage specified based at least in part on the card type associated with a card. The card type may be indicated by at least a portion of the card number of a card. Or the card type may be indicated by one or more of: a magnetic stripe of a card, a radio frequency identification (RFID) chip of a card, a near field communication (NFC) chip of a card, a card network associated with a card, or an issuer associated with a card, etc.

In some embodiments, the system 200 communicates information relating to the second card transaction or the refund for the second amount to the first medical provider. For example, the system may generate a report for each medical provider regarding the discounts that were processed through the discount network.

In some embodiments, in response to determining that the discount program includes two or more discounts, the system 200 performs the second card transaction or provides the refund by determining whether at least one of the two or more discounts is applicable to the card transaction. In response to determining that at least one of the two or more discounts is applicable to the card transaction, the system 200 performs the second card transaction or provides the refund for the second amount.

The routine 400 can include fewer, more, or different blocks than those illustrated in FIG. 4 without departing from the spirit and scope of the description. Moreover, it will be appreciated by those skilled in the art and others that some or all of the functions described in this disclosure may be embodied in software executed by one or more processors of the disclosed components and mobile communication devices. The software may be persistently stored in any type of non-volatile and/or non-transitory storage.

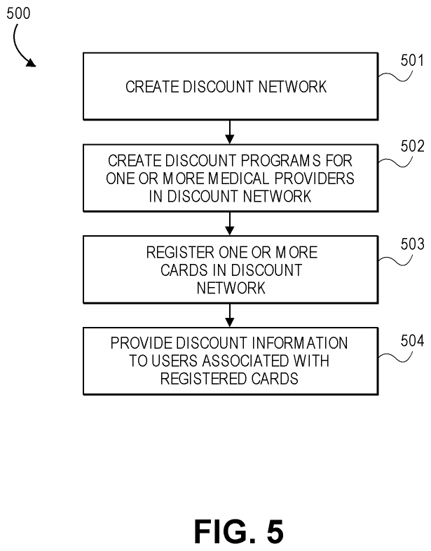

FIG. 5 is a flow diagram illustrative of a routine 500 for providing discount programs for medical services, according to certain embodiments. The routine 500 is described with respect to the system 300 of FIG. 3. However, one or more of the steps of routine 500 may be implemented by other systems, such as those described in greater detail above with reference to FIGS. 1 and 2. The routine 500 can be implemented by any one, or a combination of, components in the system 300. Further details regarding certain aspects of at least some of steps of the routine 500 are described in greater detail above with reference to FIG. 3.

At block 501, the system 300 creates a discount network. The discount network may include discount programs for medical providers. Information relating to the discount programs and the discount network may be stored in one or more data structures in one or more databases (e.g., the provider/discount database 310).

At block 502, the system 300 creates discount programs for one or more medical providers in the discount network. Each of the one or more medical providers may be identified by a unique identifier of a card reader associated with respective medical provider. Each discount program can specify at least one discount for medical procedures provided by the respective medical provider and can be provided by an agreement between the discount network and the respective medical provider.

In some embodiments, the system 300 provides a provider interface to receive user input for creating a discount program, the user input including the unique identifier of the card reader associated with a medical provider and at least one discount percentage. The provider interface may be one or more of: a web page, an application, a mobile app, an email interface, a phone interface, a facsimile interface, etc. The provider interface may be the same as or similar to the provider interface 140 in FIG. 1.

At block 503, the system 300 registers one or more cards in the discount network. A card may be one or more of: a credit card, a debit card, a gift card, a general purpose reloadable (GPR) card, a single use card, a virtual card, a closed network card, etc. Information relating to users and the one or more card numbers may be stored in the one or more data structures in one or more databases (e.g., the user/card number database 320).

At block 504, the system 300 provides discount information to users associated with the registered cards. For example, the system 300 provides a user interface to display the discount programs to users associated with the one or more cards registered in the discount network. The discount in each discount program may be applied in two separate card transactions, a first card transaction to add an amount charged for a medical procedure provided by the respective medical provider to an account of the respective medical provider and a second card transaction to subtract an amount reflecting the discount from the account of the respective medical provider, the first card transaction performed using a card of the one or more cards registered in the discount network. The system 300 may perform the second card transaction automatically subsequent to the first card transaction. Transaction information relating to the first card transaction may be received from a transaction processing system. The amount reflecting the discount can be determined by accessing one or more databases subsequent to the first card transaction.

In some embodiments, the system 300 adds the amount reflecting the discount to an account associated with the card used in the first card transaction. In other embodiments, the system 300 provides a refund for the amount reflecting the discount to a user associated with the card used in the first card transaction. The refund can be one or more of: points associated with a reward program, mileage associated with a reward program, a cash back amount, a check, a gift card, etc.

In one embodiment, the discount in a discount program is a discount percentage associated with the unique ID of the card reader associated with a medical provider, the discount percentage to be applied to the amount charged in the first card transaction. In another embodiment, the discount in a discount program is a discount percentage specified based at least in part on a card type associated with a card. The discount in a discount program can be eligible to be changed at a specified interval, wherein the specified interval is a week, two weeks, a month, etc. For instance, a medical provider can update the discount every week, every two weeks, every month, etc. as appropriate.

The user interface may be one or more of: a web page, an application, a mobile app, an email interface, a phone interface, a facsimile interface, etc. In certain embodiments, the system 300 registers the one or more cards in the discount network by receiving user input through the user interface, the user input including a card number of the one or more cards. The user interface can be the same as or similar to the user interface 150 in FIG. 1.

The routine 500 can include fewer, more, or different blocks than those illustrated in FIG. 5 without departing from the spirit and scope of the description. Moreover, it will be appreciated by those skilled in the art and others that some or all of the functions described in this disclosure may be embodied in software executed by one or more processors of the disclosed components and mobile communication devices. The software may be persistently stored in any type of non-volatile and/or non-transitory storage.

The various illustrative processes described herein may be implemented as electronic hardware, computer software, or combinations of both. To clearly illustrate this interchangeability of hardware and software, various illustrative components, blocks, modules, and states have been described above generally in terms of their functionality. However, while the various modules are illustrated separately, they may share some or all of the same underlying logic or code. Certain of the logical blocks, modules, and processes described herein may instead be implemented monolithically.

The various processes described herein may be implemented or performed by a machine, such as a computer, a processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor may be a microprocessor, a controller, microcontroller, state machine, combinations of the same, or the like. A processor may also be implemented as a combination of computing devices, e.g., a combination of a DSP and a microprocessor, a plurality of microprocessors or processor cores, one or more graphics or stream processors, one or more microprocessors in conjunction with a DSP, or any other such configuration.

The processes described herein may be embodied directly in hardware, in a software module executed by a processor, or in a combination of the two. For example, each of the processes described above may also be embodied in, and fully automated by, software modules executed by one or more machines such as computers or computer processors. A module may reside in a computer-readable storage medium such as RAM memory, flash memory, ROM memory, EPROM memory, EEPROM memory, registers, hard disk, a removable disk, a CD-ROM, memory capable of storing firmware, or any other form of computer-readable storage medium known in the art. An exemplary computer-readable storage medium can be coupled to a processor such that the processor can read information from, and write information to, the computer-readable storage medium. In the alternative, the computer-readable storage medium may be integral to the processor. The processor and the computer-readable storage medium may reside in an ASIC.

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes. Moreover, in certain embodiments, acts or events may be performed concurrently, e.g., through multi-threaded processing, interrupt processing, or via multiple processors or processor cores, rather than sequentially.

Conditional language used herein, such as, among others, “can,” “could,” “might,” “may,” “e.g.,” and from the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

While the above detailed description has shown, described, and pointed out novel features as applied to various embodiments, it will be understood that various omissions, substitutions, and changes in the form and details of the logical blocks, modules, and processes illustrated may be made without departing from the spirit of the disclosure. As will be recognized, certain embodiments of the inventions described herein may be embodied within a form that does not provide all of the features and benefits set forth herein, as some features may be used or practiced separately from others.

What is claimed is:

1. A method of providing discounted prices for medical services, the method comprising:
using one or more computing devices comprising computer hardware:
creating a discount network that includes discount programs for medical providers;
creating discount programs for one or more medical providers in the discount network, wherein each of the one or more medical providers is identified by a unique identifier of a card reader associated with respective medical provider, and wherein each discount program specifies at least one discount for medical procedures provided by the respective medical provider and is provided by an agreement between the discount network and the respective medical provider, information relating to the discount programs and the discount network stored in one or more data structures in one or more databases;
registering one or more cards in the discount network, information relating to the one or more card numbers stored in the one or more data structures in the one or more databases; and
providing a user interface to display the discount programs to users associated with the one or more cards registered in the discount network,
wherein the discount in each discount program is applied in two separate card transactions, a first card transaction to add an amount charged for a medical procedure provided by the respective medical provider to an account of the respective medical provider and a second card transaction to subtract an amount reflecting the discount from the account of the respective medical provider, the first card transaction performed using a card of the one or more cards registered in the discount network, the second card transaction performed automatically subsequent to the first card transaction, transaction information relating to the first card transaction received from a transaction processing system, the amount reflecting the discount determined by accessing the one or more databases subsequent to the first card transaction.
